(19) United States
(12) Patent Application Publication
(10) Pub. No.: US 2004/0106644 A1
(43) Pub. Date: Jun. 3, 2004

(54) Composition for treatment of night sight problems(halos, comas and glare) after refractive surgery, intra ocular lens implant after lensectomy or intraocular implant in phakic patients comprising aceclidine employed at low concentrations

(76) Inventor: Alessandro Randazzo, Milano (IT)

Correspondence Address: SUGHRUE MION, PLLC, 2100 PENNSYLVANIA AVENUE, N.W., SUITE 800, WASHINGTON, DC, 20037, US

(21) Appl. No.: 10/473,740

(22) PCT Filed: Mar. 29, 2002
(86) PCT No.: PCT/EP02/03542

(30) Foreign Application Priority Data
Apr. 3, 2001 (IT) MI2001A000708

(51) Int. Cl. A61K 31/4745

(52) U.S. Cl. 514/305

(57) ABSTRACT

After refractive surgery to reduce ametropy (i.e. myopia, astigmatism or hypermetropia) an average percentage of patients between 15.8% after PRK (Photo Refractive Keratectomy) and 33% after LASIK (Laser in situ Keratomileusis) shows a poor night sight due to the presence of halos, glare and coma. A comparable disorder is present in a percentage of patients that underwent lensectomy (cataract or refractive lensectomy) with intra ocular lens (IOL) implant and IOL implants in phakic patients to reduce ametropy. Thanks to the effect on pupillary kinetics, diluted low concentrations (from 0.002% to 0.040%) of Aceclidine were surprisingly found to effectively reduce and/or eliminate night sight problems for about 6 hours.

### BACKGROUND OF THE INVENTION

1. Field of the Invention

The present invention relates to the treatment of night sight problems like halos, coma and glare to which are exposed a percentage of patients who underwent refractive surgery or lensectomy with intra ocular lens (IOL: monofocal, multifocal, etc) implant in aphakic patients or intra ocular lens implant in phakic patients (“phakic IOL” like intra chamber lens ICL; Artisan, NuVita etc.). The treatment generally relates to the ophthalmic use of a pharmaceutical composition, in particular ophthalmic composition, containing aceclidine (3-acetoxyquinuclidine, R. Paoletti et al., 1998), at very low concentrations.

A highly preferred concentration range of aceclidine in total weight percent is from 0.002% to 0.040%, more preferably from 0.016 to 0.032% (of total weight percent of composition).

After refractive surgery to reduce ametropy (like for example myopia, astigmatism or hypermetropia), an average percentage of patients between 15.8% after PRK (Photo Refractive Keratectomy) and 33% after LASIK (Laser in Situ Keratomileusis) suffers of severe night sight problems due to light ray aberrations and diffraction (Rossetti et al, 2001). A comparable disorder is present in a percentage of patients that underwent lensectomy with intra ocular lens (IOL) implant (cataract or refractive lensectomy) (Martin L et al 1999; Schmitz S et al 2000; Pieh S et al 2001; Hwang IP et al 2001; Walkow L et al 2001) or with intra ocular lens implants in phakic patients to reduce ametropy (Maroccos R et al 2001).

Nowadays PRK and LASIK are the most important surgery techniques to reduce ametropy (Pop et al, 1999; Clinch et al, 1999; El-Maghraby et al, 1999; El Danasoury et al, 1999; Hersh et al, 1997). PRK consists in the laser ablation of the cornea stroma surface by use of Excimer Laser after epithelial cell removal. LASIK uses the same procedure of PRK after having created a corneal flap with a microkeratom. Radial keratotomy (RK) is an older procedure used to treat myopia. This procedure involves making radial incisions in the cornea with a diamond blade. The number and depth of radial incisions corresponds with the amount of desired correction.

These techniques do not treat the whole anterior corneal surface but they concentrate on a central optical zone with different diameters depending on corneal thickness and starting ametropy. For this reason, after surgery the “useful” portion of the cornea for a good sight is the most central one (O'Brart et al, 1995). Thus, the transition area between the treated and not treated zones may cause diffraction and aberration phenomena during night hours when pupil is typically midriatic (Martinez et al, 1998; Hersh et al, 2000). During daylight hours, since the pupil diameter is smaller than or equal to the treated optical zone, diffractions and aberrations are generally absent.

Comparable problems of light ray aberrations and diffraction are typically present in lensectomized patients with IOL implants of small diameter, because of difference between IOL diameter and mesopic pupillary diameter. Therefore, lensectomized patients may also have poor night sight due to the presence of halos, glare and coma as well.

Recently a new surgery technique has been developed for the treatment of high level ametropy that keeps the patient's lens. It consists in the insertion of a IOL in front of patient's lens either in the anterior or in the posterior chamber through a small incision (“phakic IOL” like ICL; Artisan, NuVita etc). This surgical technique may cause night vision problems as well.

Alteration of night sight causes difficulties in normal night activities (i.e. driving).

### SUMMARY OF THE INVENTION

The present invention solves the problem of light ray diffraction and aberration during night hours. Very surprisingly we found that the administration of an ophthalmic composition containing aceclidine, in very low concentrations, may effectively reduce the pupillary diameter for a period of up to six hours. A sufficient fluid amount of an ophthalmic composition in accordance with the present invention may be instilled in order to cause pupil diameter reduction of more than 2-3 mm for a time period of four to six hours. This is an important feature of the present invention and enables the treatment of halos, coma and glare following refractive surgery (i.e. RK, PRK, LASIK). It may further provide means for holding and supporting IOL implants in phakic or aphakic patients following lensectomy or IOL refractive implants.

For decades parasympathomimetic compounds (like for example aceclidine, pilocarpine or carbachol) have been used at concentration of 2 or more percent by weight to reduce intra ocular pressure in glaucoma patients. These compounds enhance the aqueous humour outflow by contracting the ciliary muscle that makes traction on scleral spur widening the trabecular meshwork. They induce myosis stimulating iris muscles, contract the ciliary muscle causing forward movements of the lens with increasing myopia, lens thickening and decreasing depth of the anterior chamber. These compounds have however several serious side effects such as:

1) drug dosage dependent myosis, with narrowing of peripheral isopter of the visual field;

2) ciliary muscle contraction, which may cause headache;

3) myopia enhancement lasting about 60-90 minutes after instillation, strictly related to the concentration;

4) decreasing depth of the anterior chamber with possible retina breaks due to peripheral traction, dosage dependent and/or in case of patient predisposition;

5) loss of accommodative ocular reaction of the treated eye.

For the reasons stated above, parasympathomimetic compounds (e.g. aceclidine, pilocarpine, carbachol) have not been suggested nor used in their original commercial concentration to reduce night sight problems after refractive surgery. The present invention provides now aceclidine in very low concentration for the treatment of the addressed problem. Surprisingly, it is found that such very low concentrations of aceclidine can provide fairly specifically the possibility to exploit iris sphincter muscle contractions inducing the natural nocturnal mydriasis and eliminating light ray aberrations and diffraction, namely halos, coma and glare, without the undesired side effects of the drugs as indicated above.

Aceclidine was found to be in particular useful to increase pupillary myosis and act on intra ocular pressure and ciliary body less than other compounds, reducing sight adaptation problems and retina breaks due to peripheral traction (R. Paoletti et al., 1998).

The effective dilution range is in particular from 0.016% to 0.040% of total weight percent of an ophthalmic composition. The diluted compound may be instilled in the eye typically 10-20 minutes before need and it acts for about 4-6 hours. In the above aceclidine concentration range no relevant side effects are detectable Aceclidine may also be useful in even lower concentrations such as 0.002% weight (of total weight of an ophthalmic composition). Accordingly aceclidine may typically be used from 0.002%-0.016%, preferably from 0.016-0.032%, also preferably from 0.032-0.040%. Aceclidine is typically formulated in an aqueous solution being adapted to ophthalmic administration. Accordingly, such an ophthalmic composition may also comprise tonicity enhancers, such as sodium chloride, glycerol, boric acid, sorbitol, mannitol and the like. It may also comprise a pH-adjusting agent to adjust the pH of the final ophthalmic composition to a physiological pH. Such pH-adjusting agents are typically a buffer such as a phosphate buffer, sodium acetate, boric acid, ammonium chloride, and the like. Such excipients are known in the art and may be used as appropriate.

The above ophthalmic compositions comprising aceclidine in very low concentrations were tested on patients treated with refractive surgery who reported a statistically significant improvement (sometimes also total regression) of halos, coma and glare and a good night sight improvement after instillation of the ophthalmic composition. Reported side effects are light, transitory (5-10 minutes) conjunctival hyperemia, while no headache or sight reduction are present.

Another surprising finding is the high selectivity of aceclidine as compared to other parasympathomimetic drugs. This selectivity addresses the effective reduction/prevention of halos, coma and glares in patient who had refractive surgery, as described above, wherein virtually no side effects (as described above) are detectable. Accordingly, aceclidine is strongly preferred over pilocarpine and carbachol.

### BRIEF DESCRIPTION OF THE FIGURES

FIG. 1 shows the four graduated images used for the objective determination of the coma grading by the patients.

FIG. 2 shows the four graduated images used for the objective determination of the halos grading by the patients.

FIG. 3 shows the pupillary diameter variations average of a healthy people group treated with two aceclidine ophthalmic compositions at two different concentrations; the determination of the pupillary diameter has been performed after 30 minutes, 1, 2, 3, 4, 5 and 6 hours following the instillation of the I (4 IU, 0.016%) and II (8 IU, 0.032%) dilution of the aceclidine ophthalmic compositions.

### DETAILED DESCRIPTION

The following clinical trail has been performed in order to demonstrate the activity of aceclidine at different concentrations on patients who underwent refractive surgery.

A double-masked randomized clinical trial with 14 patients (27 eyes) has been organized by dividing the patients in three different groups: 8 patients were treated with placebo, 10 patients were treated with a first ophthalmic composition (I dilution), 9 patients were treated with a second ophthalmic composition (II dilution).

The patients belonging to this trial had to go through an ophthalmic examination: of the haze grading and of natural and corrected visual acuity, intra ocular pressure (IOP), pupillometry in mesopic conditions with split lamp and examination of corneal maps. The anamnestic data about the surgical operation were collected: the refractive surgery technique employed (PRK/LASIK), the date of the surgical operation, data about the corrected optical zone and the ametropy kind. The night sight problems had to be stable for at least three months after the surgical operation for the patient to be included in the trial.

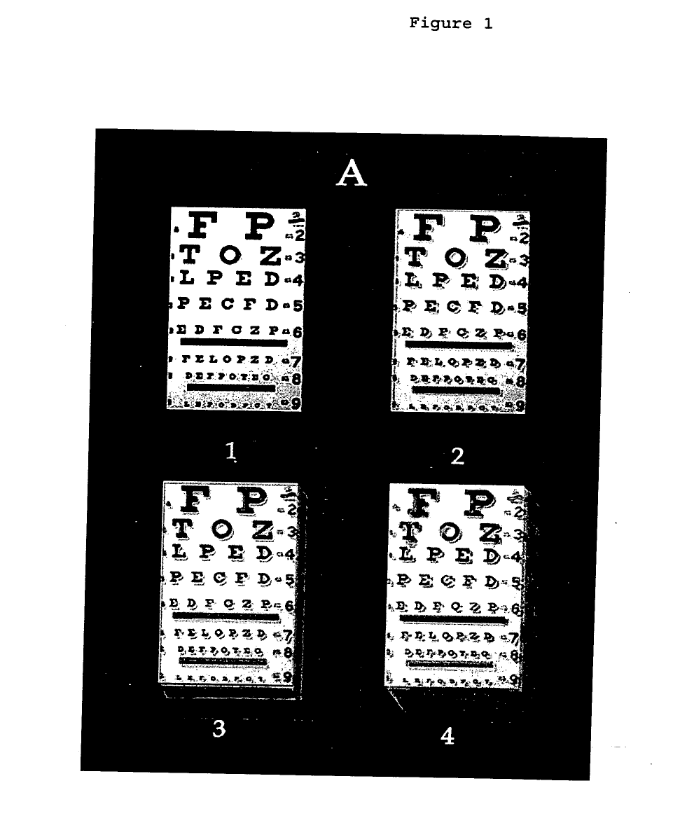

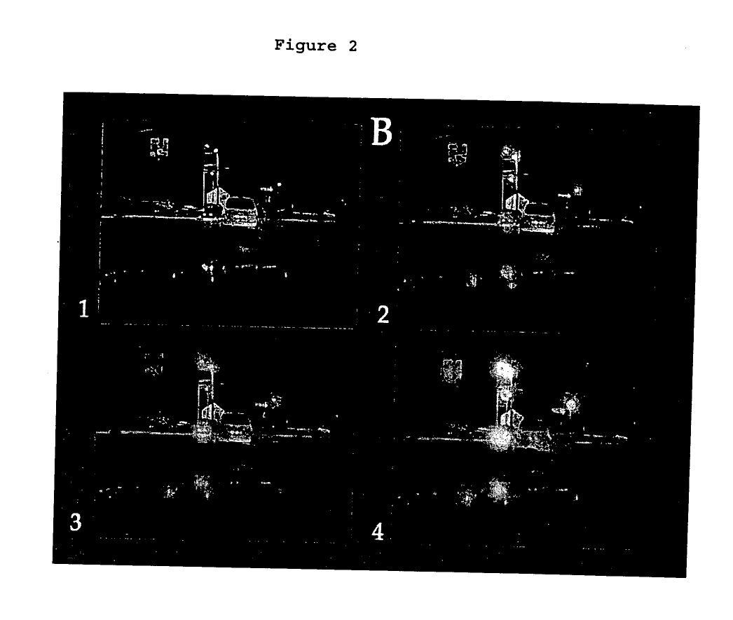

In order to render the clinical trial as much as possible objective and reproducible, the values of halos and coma perceived by the patients were graduated using eight images got from the web site www.surgical.com and elaborated with the Photoshop program; a value of halos and coma (on a scale from 1 to 4) was assigned to each image, as showed in FIGS. 1 and 2.

The patients had to identify the image they perceived before and during the pharmacological treatment, with follow-up examinations fixed every 15-30 days. At the end of the clinical trial the patients had to provide information about the efficacy and duration (in terms of hours) of the treatment, onset time of the effect, changes of the visual capabilities and side effects, if any.

The ophthalmic compositions were prepared according to the following procedure:

I dilution, 4 IU of aceclidine 2% were diluted in 5 mL of hyaluronic acid (0.200 g/100 mL)

II dilution, 8 IU of aceclidine 2% were diluted in 5 mL of hyaluronic acid (0.200 g/100 mL)

Placebo consisted in 5 mL of hyaluronic acid (0.200 g/100 mL).

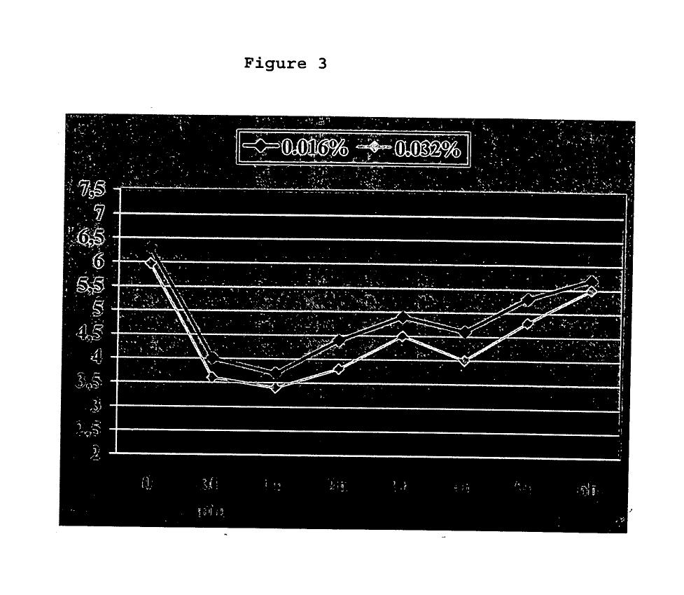

The two ophthalmic compositions, I and II dilution, were administered firstly to a group of healthy people: one eye was treated with the I dilution composition (0.016%), the second eye with the II dilution composition (0.032%), measuring the pupillary diameter in the same light conditions at 30 minutes, 1-2-3-4-5-6 hours after the first instillation. FIG. 3 shows the behavior of the pupillary diameter. A mean reduction of the pupillary diameter of 2.5 mm is showed within the first 30 minutes following the instillation and with a trend to disappear after 5-6 hours.

The three ophthalmic compositions (I dilution, II dilution and placebo) were administered to the 14 patients belonging to the clinical trial in a blind way.

The results showed that 18 out of 19 eyes treated with diluted aceclidine ophthalmic compositions (I and II dilution) versus 2 out of 8 treated with placebo showed an improvement in night vision problems (95% vs 25%; p=0.04).

The efficacy in terms of hours has been reported to 6 hours with the onset after 15-20 minutes following the instillation. Mean improvement was 1.4 (±0.6) for coma and 1.14 (±0.4) for halos; 61% of patients reported a night vision acuity improvement.

The reported side effect is a modest and transient conjunctival hyperemia lasting for about 10-15 minutes. None reported headache, myopia enhancement or other alterations. Moreover no differences have been reported on the IOP or the pupillary kinetics during the follow-up examinations.

### BIBLIOGRAPHY

1. Paoletti R., Nicosia S., Clementi F., Fumagalli G., in Farmacologia oculare, a cura di: Filippo Drago e Nicola Orzatesi, ed. UTET, 1998.

2. Rossetti L., Randazzo A., Fogagnolo P., Orzalesi N., Comparison of PRK vs LASIK for correction of myopia. The results of meta-analysis of published literature, ARVO 2001.

3. Martin L. Computerized method to measure glare and contrast sensitivity in cataract patients, J. Cataract Refract. Surg. 1999 Mar; 25(3):411-5.

4. Schmitz S, Dick H B, Krummenauer F, Schwenn O, Krist R. Contrast sensitivity and glare disability by halogen light after monofocal and multifocal lens implantation. Br. J. Ophthalmol. 2000 October; 84(10):1109-12.

5. Pieh S, Lackner B, Hanselmayer G. Zohrer R, Sticker M, Weghaupt H, Fercher A, Skorpik C. Halo size under distance and near conditions in refractive multifocal intraocular lenses. Br. J. Ophthalmol. 2001 July;85(7):816-21.

6. Hwang I P, Olson R J. Patient satisfaction after uneventful cataract surgery with implantation of a silicone or acrylic foldable intraocular lens. Comparative study. J. Cataract Refract. Surg. 2001 October; 27(10):1607-10.

7. Walkow L, Klemen U M. Patient satisfaction after implantation of diffractive designed multifocal intraocular lenses in dependence on objective parameters. Graefes Arch Clin. Exp. Ophthalmol. 2001 September;239(9):683-7.

8. Maroccos R, Vaz F, Marinho A, Guell J, Lohmann C P., Glare and halos after “phakic IOL”. Surgery for the correction of high myopia Ophthalmologe 2001 November; 98(11):1055-9.

9. Pop M, Payette Y. Multipass versus single pass photorefractive keratectomy for high myopia using a scanning laser. J. Refract. Surg. 1999 July-August;15(4):444-50.

10. Clinch T E, Moshirfar M, Weis J R, Ahn C S, Hutchinson C B, Jeffrey J H. Comparison of mechanical and transepithelial debridement during photorefractive keratectomy. Ophthalmology 1999 March;106(3):483-9.

11. El-Maghraby A, Salah T, Waring GO 3rd, Klyce S, Ibrahim O. Randomized bilateral comparison of excimer laser in situ keratomileusis and photorefractive keratectomy for 2.50 to 8.00 diopters of myopia. Ophthalmology 1999 March;106(3):447-5.

12. El Danasoury Mass., El-Maghraby A, Klyce S D, Mehrez K. Comparison of photorefractive keratectomy with excimer laser in situ keratomileusis in correcting low myopia (from −2.00 to −5.50 diopters). A randomized study. Ophthalmology 1999 February 106(2):411-20; discussion 420-1.

13. Hersh P S, Stulting R D, Steinert R F, Waring G O 3^{rd}, Thompson K P, O'Connell M, Doney K, Schein O D. Result of phase III excimer laser photorefractive keratectomy for myopia. The Summit PRK Study Group. Ophthalmology 1997 October; 104(10):1535-53.

14. O'Brart D P, Corbett M C, Lohmann C P, Kerr Muir M G, Marshall J. The effects of ablation diameter on the outcome of excimer laser photorefractive keratectomy. A prospective, randomized, double-blind study. Archives of Ophthalmology 1995 April 113(4):438-43.

15. Martinez, Carlos E. M S, M D; Applegate, Raymond A. O D, Ph D et al. Effect of pupillary dilatation on corneal optical aberration after photorefractive keratectomy. Arch. Ophthalmology, Vol 116(8) August 1998; 1053-1062.

16. Hersh P S, Steinert R F, Brint S F. Photorefractive keratectomy versus laser in situ keratomileusis: comparison of optical side effects. Ophthalmology 2000 May;107(5):925-33.

1. Use of aceclidine or a pharmaceutically acceptable derivative thereof in the manufacture of a composition for the treatment of night sight problems (i.e. halos, coma and glare) in patients who underwent refractive surgery or intra ocular phakic lens implant or intra ocular implant in aphakic patients.

2. The use according to claim 1 wherein the concentration range of aceclidine is from 0.002% to 0.040% (weight % of total concentration), preferably from 0.002% to 0.016% or from 0.016% to 0.040% and in particular from 0.016% to 0.032%.

3. Pharmaceutical composition for the treatment of night sight problems (i.e. halos, coma and glare) in patients who underwent refractive surgery or intra ocular phakic lens implant or intra ocular implant in aphakic patients comprising aceclidine or a pharmaceutically acceptable derivative thereof and a pharmaceutically acceptable carrier.

4. Pharmaceutical composition according to claim 3 wherein the concentration of aceclidine is from 0.002% to 0.040% (weight % of total concentration), preferably from 0.002% to 0.016% or from 0.016% to 0.040% and in particular from 0.016% to 0.032%.

5. Pharmaceutical composition according to claim 3 or 4 wherein the pharmaceutically acceptable carrier is hyaluronic acid.

6. Process for preparing a composition according to claim 3 which comprises bringing aceclidine or a pharmaceutically acceptable derivative thereof into association with a pharmaceutically acceptable carrier.

7. Process according to claim 6 wherein the concentration of aceclidine is from 0.002% to 0.040% (weight % of total concentration), preferably from 0.002% to 0.016% or from 0.016% to 0.040% and in particular from 0.016% to 0.032%.

8. Process according to claim 6 or 7, wherein the pharmaceutically acceptable carrier is hyaluronic acid.

9. Method to treat night sight problems (i.e. halos, coma and glare) in patients who underwent refractive surgery or intra ocular phakic lens implant or intra ocular implant in aphakic patients, which method comprises the topical administration of an effective amount of aceclidine in an ophthalmic composition to said patient in need of such treatment.

10. Method according to claim 9, wherein the concentration of aceclidine is from 0.002% to 0.040% (weight % of total concentration), preferably from 0.002% to 0.016% or from 0.016% to 0.040% and in particular from 0.016% to 0.032%.
